(19) United States
(12) Patent Application Publication
(10) Pub. No.: US 2026/0276573 A1
(43) Pub. Date: Sep. 17, 2026

(54) SYSTEM AND METHOD FOR MEASURING PLANT WATER CONTENT AND PREDICTING WATER STRESS

(71) Applicant: The Regents of the University of California, Oakland, CA (US)

(72) Inventors: Reza Ehsani, Merced, CA (US); Arash Mohammadi Toudeshki, Merced, CA (US)

(21) Appl. No.: 19/557,437

(22) Filed: Mar. 5, 2026

Related U.S. Application Data
(60) Provisional application No. 63/770,589, filed on Mar. 12, 2025.

(51) Int. Cl. G01N 27/22; G01N 1/44; G01N 25/18; G01N 33/00; G05D 1/689; H05B 1/02

(52) U.S. Cl. CPC G01N 27/223; G01N 1/44; G01N 25/18; G01N 33/0098; G05D 1/6895; H05B 1/023

(57) ABSTRACT

An apparatus for measuring plant leaf water content includes a clamp having opposing jaws configured to secure a leaf. A sensing unit on a first jaw measures a temperature of the leaf and an electrical property of the leaf. A heating unit on a second jaw applies controlled heat to the leaf. A control unit regulates power to the heating unit, receives data indicative of a temperature response and the electrical property, and determines a water-content value for the leaf based on the data. In some embodiments, a capacitance sensor is coupled to the leaf and is interrogated to obtain the electrical property independent of the heating unit. The apparatus may be operated as a standalone clip or may be mounted to an end effector of an articulating arm of a mobile robotic platform to obtain measurements from multiple plants, and may include environmental sensing, geotagging, and wireless communication.

### CROSS-REFERENCE TO RELATED APPLICATIONS

This application claims the benefit of, and priority to, U.S. Provisional Patent Application Ser. No. 63/770,589 filed on Mar. 12, 2025. The entire contents of the foregoing application are incorporated by reference herein.

### GOVERNMENT LICENSE RIGHTS

This invention was made with Government support under Grant No. EEC-1941529, awarded by National Science Foundation. The Government has certain rights in the invention.

### BACKGROUND

Efficient water management is an important factor in modern agriculture, particularly in regions prone to drought and water scarcity. Understanding plant water status allows for optimizing irrigation practices, improving crop yield, and conserving resources. One widely recognized method for assessing plant water status is measuring stem water potential using a pressure chamber. While effective, this approach is highly invasive, time-consuming, and labor-intensive, making it impractical for large-scale implementation. Additionally, it provides only single-point measurements, limiting its ability to capture dynamic changes in plant hydration over time. The relative water content, also referred to as relative turgidity, has been recognized as a meaningful index of plant water status, offering an alternative metric for assessing hydration levels.

Monitoring tree water levels allows for effective irrigation management. Currently, midday Stem Water Potential (SWP) using a pressure chamber is regarded as one of the more established and widely accepted methods for determining plant water status and serves as the foundation for recommended irrigation scheduling. However, collecting SWP measurements is highly time-consuming and labor-intensive, making it impractical for assessing stress levels across large orchards. As a result, many growers rely on fixed irrigation schedules, which are often inefficient, or on evapotranspiration (ET)-based models, which fail to account for soil type variability, reducing overall accuracy. While traditional methods such as sap flow sensors, gravimetric techniques, and soil moisture sensors offer some advantages, they also present significant limitations in terms of labor, cost, and scalability.

Many existing water status monitoring sensors are designed for direct installation on individual trees, limiting their coverage and making large-scale deployment cost prohibitive. The low spatial resolution of the collected data prevents growers from implementing precision irrigation scheduling. Additionally, these sensors typically require solar panels, protective enclosures for electronic components, wiring, and batteries, which increase system complexity and cost. Ongoing expenses related to periodic monitoring and data collection further add to the financial burden. Moreover, conventional sensors are often restricted in their applicability, as they can only monitor trees of specific trunk sizes and are generally limited to tree crops. Some also have a limited operational lifespan, as trees may eventually reject electrodes inserted into their trunks, reducing long-term effectiveness.

Existing sensor-based solutions for direct measurement of plant water status present several additional challenges. Many are permanently affixed to individual plants or trees, restricting their applicability to a limited number of monitoring points. Installation is often cumbersome and time-consuming, and due to high energy demands, these sensors frequently rely on solar panels and large batteries, further increasing operational complexity and cost. Consequently, many growers can afford to deploy only a small number of sensors, significantly limiting their ability to assess water status variability across an entire field or orchard.

There is a need for a non-invasive, cost-effective, and scalable solution that enables rapid and widespread monitoring of plant water status. Such a system should facilitate high-resolution data collection across a large number of plants while minimizing installation complexity and operational costs. An improved approach would provide growers with real-time, actionable insights into plant hydration levels, enabling more precise irrigation scheduling and resource management.

### SUMMARY

According to one embodiment of the present disclosure, an apparatus for measuring plant leaf water content is provided. The apparatus includes a clamp having a pair of opposing jaws configured to receive and secure a leaf of the plant. A sensing unit is disposed on a first jaw and is configured to measure at least one thermal and electrical property of the leaf. A heating unit is disposed on a second jaw and is configured to apply controlled heat to the leaf. A control unit is operably coupled to the sensing unit and the heating unit, and is configured to regulate power to the heating unit, receive data corresponding to a temperature change and an electrical property of the leaf, and analyze the received data to determine the water content of the plant. The system may also include a capacitance sensor to measure the leaf water content independent from heating unit.

Implementations of this embodiment may include one or more of the following features. The clamp may include a spring-loaded mechanism to maintain a consistent clamping force on the leaf. The sensing unit may include a temperature sensor, which may be a thermocouple, a resistance temperature detector (RTD), or a thermistor. Additionally, the sensing unit may include an optical sensor in the range of 200 to 1000 nm, and/or an electrical sensor, such as a capacitance sensor, a resistive sensor, or an impedance sensor. The heating unit may include a resistive heating element. The control unit may further include an electrical battery, a power management system to regulate power distribution, a memory module for storing data, and a real-time clock for timestamping measurements. A wireless communication unit may also be included for transmitting collected data to a remote computing device. Furthermore, the apparatus may include at least one environmental sensor configured to monitor ambient temperature, humidity, atmospheric pressure, wind speed, or light intensity. The control unit may adjust heating parameters based on environmental sensor readings and compare the measured thermal and electrical responses to predefined thresholds to determine the water content of the plant.

According to another embodiment, a method for determining plant water content is disclosed. The method includes securing a plant leaf between a pair of opposing jaws of a spring-loaded clamp. The method further includes initiating a controlled heating cycle by supplying power to a heating unit disposed on one jaw, monitoring a temperature change in the leaf using a temperature sensor on the other jaw, and determining whether the temperature change has reached a predefined threshold. Once the threshold is reached, the method includes discontinuing heating, recording the temperature response, measuring an electrical property of the leaf, storing the recorded temperature response and electrical data in a memory module, and analyzing the stored data to determine plant water content. The capacitance sensor may be attached to the leaf or printed on the leaf and used as a passive sensor, and leaf water content can be read by an interrogator's unit as it passes by the sensor wirelessly.

Implementations of this embodiment may include one or more of the following features. The method may further include determining whether the temperature change is greater than zero, and stopping the controlled heating cycle if it is not. The method may also include timestamping the recorded temperature response and electrical property using a real-time clock, and geotagging the data using a GPS module.

According to another embodiment of the present disclosure, a mobile robotic platform for measuring plant water content is provided. The platform includes a mobility system configured to navigate an agricultural environment, which may include a tracked or wheeled system. A base houses a control unit responsible for processing measurement data and controlling platform operation. The platform further includes an articulating arm extending from the base, which may consist of one or more links interconnected by rotational joints, enabling the arm to position a sensing apparatus relative to a plant leaf. At the distal end of the articulating arm, the platform includes a grasper end effector for engaging plant leaves. The grasper includes a pair of opposing jaws configured to secure a plant leaf, a sensing unit on a first jaw configured to measure thermal and electrical properties, and a heating unit on a second jaw configured to apply controlled heat to the leaf. The control unit is operably coupled to the sensing and heating units and is configured to regulate power, receive data corresponding to temperature changes and electrical properties, and analyze the data to determine plant water content. In another setup, the sensor can be integrated into a grasper that can remain stationary in one location on a particular leaf and read the water content of the leaf continuously by programming the grasper to open and close in specific predetermined intervals.

Implementations of this embodiment may include one or more of the following features. The sensing unit may include a temperature sensor, which may be a thermocouple, an RTD, or a thermistor. The heating unit may include a resistive heating element. The control unit may further include an electrical battery, a power management system to regulate power distribution, a memory module for storing data, and a real-time clock for timestamping measurements. A wireless communication unit may be included to transmit collected data to a remote computing device. The mobile robotic platform may also incorporate at least one environmental sensor to monitor ambient temperature, humidity, atmospheric pressure, wind speed, or light intensity. The control unit may dynamically adjust heating parameters based on environmental readings and compare measured plant properties to predefined thresholds to determine plant water content.

### BRIEF DESCRIPTION OF DRAWINGS

Various embodiments of the present disclosure are described herein below with reference to the figures wherein:

FIG. 1 is a perspective view of an apparatus for monitoring plant leaf water content in accordance with aspects of the present disclosure;

FIG. 2 is a schematic, side view of the apparatus of FIG. 1 for monitoring plant leaf water content in accordance with aspects of the present disclosure;

FIG. 3 is a block diagram of a system including the apparatus of FIG. 1 for monitoring plant leaf water content in accordance with aspects of the present disclosure;

FIG. 4 is a flow chart of a method for monitoring plant leaf water content in accordance with aspects of the present disclosure;

FIG. 5 is a mobile robotic platform including the apparatus of FIG. 1 for monitoring plant leaf water content in accordance with aspects of the present disclosure; and

FIG. 6 is a photograph of the apparatus of FIG. 1 attached to a leaf in accordance with aspects of the present disclosure.

### DETAILED DESCRIPTION

Although this disclosure will be described in terms of specific aspects, it will be readily apparent to those skilled in this art that various modifications, rearrangements, and substitutions may be made without departing from the spirit of this disclosure. For the purpose of promoting an understanding of the principles of this disclosure, reference will now be made to exemplary aspects illustrated in the drawings, and specific language will be used to describe the same. It will nevertheless be understood that no limitation of the scope of this disclosure is thereby intended. Any alterations and further modifications of the features illustrated herein, and any additional applications of the principles of this disclosure, as illustrated herein, which would occur to one skilled in the relevant art and having possession of this disclosure, are to be considered within the scope of this disclosure.

The present disclosure provides a system and method for non-invasive measurement of plant leaf water content to assess and predict plant water stress. The system enables high-intensity, plant-specific, and rapid monitoring of relative water content, facilitating improved irrigation management. By providing real-time insights into plant hydration levels, the system supports precision irrigation scheduling, reducing water consumption without adversely affecting crop yield. The system is adaptable for use by both row crop and tree crop growers, as well as crop consultants.

The system includes an apparatus that can be directly attached to a plant for continuous monitoring or integrated into a mobile robotic platform to assess water status variability across multiple plants within a field. The collected data can be utilized to generate plant water status maps, enhancing the efficiency of irrigation practices. Additionally, the system may be incorporated into automated variable rate irrigation or deficit irrigation systems, optimizing water use based on real-time plant conditions. Beyond agricultural applications, the system can also be employed for environmental monitoring, such as assessing leaf water status in trees to help predict wildfire risk.

The disclosed apparatus is designed to address the limitations of existing water status sensors and advance the state-of-the-art in plant water status assessment. The objective is to provide a commercially viable, non-destructive (i.e., minimally invasive) sensor that delivers precise, real-time information on plant hydration levels. By offering growers improved data on plant water stress, the system enables more efficient water resource utilization, contributing to sustainable agricultural practices and improved irrigation management.

The system may be powered by a rechargeable battery or by the robotic platform that carries the apparatus and collects data from multiple trees in an orchard. A key advantage of the disclosed system over commercially available alternatives is its ability to collect data from both tree crops and row crops. Existing sensors are typically limited to tree crops and require direct attachment, making them unsuitable for broader applications in precision agriculture.

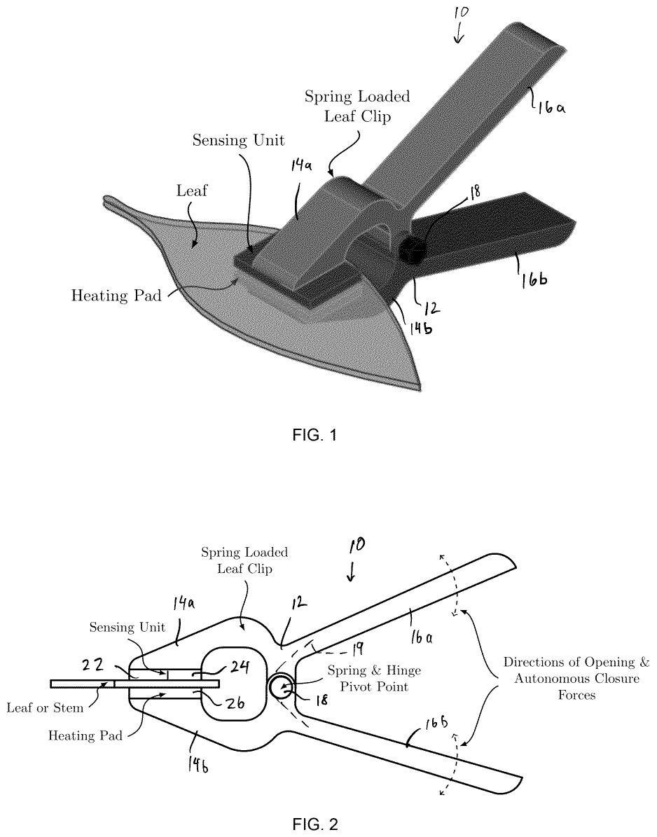

Referring to FIGS. 1 and 2, an apparatus 10 for monitoring plant leaf water content is shown. The apparatus 10 includes a spring-loaded clip or clamp 12, which securely holds a leaf or stem in place for measurement. The clamp 12 includes two opposing jaws 14a and 14b, which are configured to apply a consistent clamping force to the leaf or stem. The jaws 14a, 14b extend from respective arms 16a, 16b, which function as handles for opening and closing the clamp 12. The arms 16a, 16b are pivotally connected at a pivot pin 18, allowing them to rotate about a central axis. A spring 19 (FIG. 2) is positioned at the pivot pin 18, providing an autonomous closing force that ensures a secure grip on the leaf or stem.

A sensing unit 20 is mounted on the first jaw 14a and is configured to measure temperature and one or more electrical properties (e.g., resistance, capacitance, impedance, etc.) of the leaf. Opposing the sensing unit 20, a heating unit 26 is disposed on the second jaw 14b. The heating unit 26, which may be shaped as a heating pad, applies a controlled amount of heat to the leaf, allowing for precise thermal and electrical measurements to determine the rate of heat dissipation through the leaf tissue.

The heating unit 26 operates by converting electrical energy into thermal energy to generate controlled heat for leaf water content measurement. The heating unit 26 may include a resistive heating element, which increases in temperature when an electric current passes through it. The resistive heating element may be a trace on a rigid printed circuit board (PCB) or a flexible PCB attached to the second jaw 14b.

The sensing unit 20 may be implemented as a rigid or a flexible PCB attached to the first jaw 14a. The sensing unit 20 may incorporate multiple sensors that can include temperature sensors 22 and electrical sensors 24 (FIG. 2) to measure the heat dissipation and electrical properties of the leaf, respectively. The temperature sensors 22 function in conjunction with the heating unit 26, which applies a controlled heat pulse to the leaf, allowing the sensing unit to determine how quickly heat is transferred through the leaf. Several types of temperature sensors can be used, including, but not limited to, thermocouples, which generate a voltage proportional to temperature when exposed to heat; resistance temperature detectors (RTDs), which vary their resistance with temperature and provide high accuracy; thermistors, which are ceramic-based sensors with a strong temperature-dependent resistance; and the like.

In addition to temperature measurement, the sensing unit 20 includes electrical sensors 24 to evaluate the dielectric and conductive properties of the leaf, which are also influenced by water content. Different types of electrical sensors 24 can be integrated, including capacitance sensors, which measure the dielectric constant of the leaf tissue and detect changes in stored charge; and resistive or conductivity sensors, which assess the electrical resistance of the leaf and provide insight into ionic conductivity related to hydration levels. Since water has a high dielectric constant and is a conductive medium, changes in a leaf's capacitance or resistance directly correspond to its hydration level. A well-hydrated leaf will exhibit a higher dielectric constant and lower resistance due to greater ionic conductivity, whereas a dry leaf will show a reduced dielectric response and increased resistance. Each of these sensors 22 and 24 provides valuable data on how the leaf absorbs and dissipates heat, which correlates with the plant's hydration level. Hydrated leaves exhibit higher thermal conductivity and release heat more efficiently, whereas dehydrated leaves retain heat longer due to lower water content.

In some embodiments, a capacitance sensor may be used to obtain a leaf-related electrical signal that is processed as an indicator of leaf hydration or leaf water content. For example, the capacitance sensor may be attached to the leaf, printed on the leaf, integrated into a substrate positioned adjacent the leaf, and/or otherwise coupled to the leaf to produce a measurable electrical response. The capacitance sensor may be read by the sensing unit 20 and communicated to the controller 50, and/or the capacitance sensor may be configured as a passive structure whose response is read by an interrogator unit, including a wireless interrogator, to obtain a capacitance-related measurement. In such embodiments, the capacitance sensor output may be used alone or in combination with thermal-response information generated using the heating unit 18, and the capacitance sensor may be read independently from operation of the heating unit 18 to provide a supplemental measurement, a baseline measurement, and/or a verification measurement.

During operation, the sensing unit 20 collects temperature and electrical data simultaneously. When the apparatus 10 is clamped on a leaf, the heating unit 26 applies a controlled heat, which may be continuous or periodic, while the temperature sensor 22 measures how quickly the leaf absorbs and dissipates heat. At the same time, the electrical sensor 24 captures variations in capacitance or resistivity, allowing for a comprehensive assessment of the plant's water content. This dual-sensing approach improves accuracy and reliability by combining two complementary measurement techniques.

The clamp 12, jaws 14a, 14b, and arms 16a, 16b may be made of any durable material such as plastic, metal, or composite materials, ensuring structural integrity while minimizing interference with the sensing elements. The pivot pin 18 may be composed of metal or reinforced polymer, providing a robust rotational axis for the clamp's operation. The sensing unit 20 and heating unit 26 are secured onto their respective jaws using adhesives, fasteners, or integrated molding techniques, ensuring consistent alignment and functionality.

In operation, the apparatus 10 is clamped onto a leaf or stem, with the sensing unit 20 and heating unit 26 engaging opposite sides of the leaf. The heating unit 26 outputs heat in a controlled manner, while the sensing unit 20 records the resultant temperature changes and capacitance variations. These measurements provide data on the water content of the leaf, which can be used to assess the plant's hydration status and water stress levels. The spring-loaded mechanism ensures that the device maintains consistent contact pressure throughout the measurement process, improving the accuracy and repeatability of the readings.

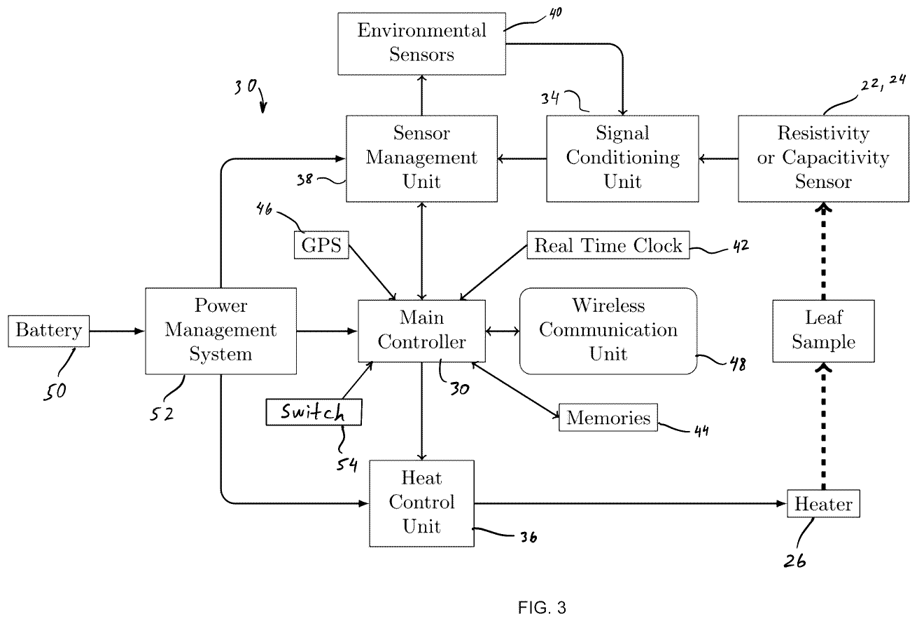

Referring to FIG. 3, a block diagram illustrates the electronic and computational components of a control unit 30 of the apparatus 10 for monitoring plant leaf water content. The control unit 30 includes various interconnected components that function together to measure, process, and communicate plant hydration data. Thus, the control unit 30 serves as the central processing hub that integrates all subsystems, manages data acquisition, regulates thermal application, and oversees system control. In addition, the control unit 30 executes programmed instructions to process sensor inputs, store data, and communicate with external devices.

The control unit 30 includes a main controller 32, which functions as the apparatus's microprocessor, executing firmware that controls measurement operations. The main controller 32 may be any suitable processor adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted by using any logic processor (e.g., control circuit) adapted to execute algorithms, calculations, and/or set of instructions described herein.

The main controller 32 receives signals from the sensors 22 and 24, processes thermal and capacitance data, regulates the heating unit 26, and determines when and how measurements should be taken. The main controller 32 also manages timing operations using the real-time clock 42 and coordinates data storage in the memory module 44. The real-time clock 42 provides timestamping functionality, allowing for precise tracking of measurement trends over time. This enables users to analyze changes in plant water content at different times of the day or under varying environmental conditions.

The memory module 44 may be non-transitory computer-readable medium configured to store any suitable computer data, such as software instructions executable by the main controller 32. The memory module 44 may also include transitory memory for loading instructions and other computer readable data during execution of the instructions. The memory module 44 may include one or more of volatile, non-volatile, such as read-only memory (ROM), random access memory (RAM), electrically erasable programmable ROM (EEPROM), non-volatile RAM (NVRAM), or flash memory. The memory module 44 stores collected data, system logs, and calibration settings. This ensures that historical data can be retrieved for trend analysis, enabling long-term monitoring of plant hydration status. The main controller 32 manages data writing and retrieval.

The electrical sensor 24, which may be a resistivity or capacitance sensor, directly interacts with the leaf sample to measure changes in electrical properties. These properties are influenced by the water content of the leaf, providing key data for hydration assessment. The sensors 22 and 24 send raw signals to a signal conditioning unit 34 for processing. The signal conditioning unit 34 amplifies, filters, and converts the raw signals from the electrical sensor 24 into a format suitable for analysis by the main controller 32. It eliminates noise and enhances signal accuracy, ensuring that only relevant data is processed.

The heating unit 26 applies a controlled amount of heat to the leaf sample to measure its thermal response. By monitoring how the leaf absorbs and dissipates heat, the main controller 32 calculates water content. The heating unit 26 is regulated by a heat control unit 36, which ensures precise temperature application without damaging the leaf. The heat control unit 36 manages the temperature of the heating unit 26, ensuring that heat is applied consistently and within safe limits. It receives instructions from the main controller 32 and adjusts power levels accordingly to maintain optimal measurement conditions.

The main controller 32, through the heat control unit 36, regulates the heating process by modulating the applied DC voltage or current to achieve a precise temperature increase. This can be done using pulse-width modulation (PWM) or a constant voltage regulator, depending on the required heating profile. Once power is supplied, the heating unit 26 raises its temperature and that of the plant leaf. The sensing unit 20 on the opposing jaw 14a monitors the heat transfer rate by measuring temperature changes over time. The apparatus 10 maintains heating for a predetermined period or until the temperature change (Δθ) reaches a specified threshold (ψ), at which point the main controller 32 cuts power to the heating unit 26 to prevent overheating and potential damage to the leaf.

The control unit 30 may also include one or more environmental sensors 40, which monitor external factors such as ambient temperature, humidity, and atmospheric pressure. Environmental sensors 40 may include one or more of the following: ambient temperature sensors, humidity sensors, atmospheric pressure sensors, wind speed sensors, light sensors, soil moisture sensors, and the like. These parameters influence the behavior of the plant and must be considered to obtain accurate water content measurements. The data from these sensors 40 is processed alongside the leaf measurements to refine the analysis. A sensor management unit 38 acts as an interface between the electrical sensor 24 and the environmental sensors 40 and provides synchronized data collection and combines multiple sensor readings to improve accuracy. The sensor management unit 38 ensures that environmental factors are accounted for when interpreting plant hydration levels. The signal conditioning unit 34 also receives signals from the environmental sensors 40. The signal conditioning unit 34 amplifies, filters, and converts the raw signals from the environmental sensors 40 into a format suitable for analysis by the main controller 32.

The control unit 30 may further include a global positioning system (GPS) module 46, which provides geolocation data, allowing users to map water status measurements to specific locations in a field or orchard. While not essential for core system operations, this feature enhances the ability to track spatial variations in plant hydration and optimize irrigation management. Additionally, the control unit 30 may include a wireless communication unit 48, which enables remote data transmission to external computing devices, mobile application platforms, cloud storage, or remote monitoring platforms. This allows users to access real-time plant hydration data from anywhere, facilitating informed decision-making.

The control unit 30 and the apparatus 10 is powered by a battery 50, which supplies power to these components, ensuring that they can operate in field environments without reliance on external power sources. The battery provides energy for all components and is managed by the power management system 52. A power management system 52 is coupled to the battery 50 and regulates energy distribution to optimize battery usage and extend operational life. It ensures that all components receive sufficient power while preventing unnecessary drain. The control unit 30 may include energy-saving modes that activate when measurements are not actively being taken.

The power management system 52 ensures that the heating unit 26 receives stable DC power from the battery 50, optimizing energy usage to prolong operational runtime in field environments. If necessary, the apparatus 10 can adjust power delivery based on ambient temperature conditions, compensating for external factors that may affect heat dissipation.

The control unit 30 may also include a switch 54 (e.g., push button), which serves as a manual activation mechanism for initiating the test sequence. In embodiments where the apparatus 10 is operated manually, the switch 54 allows a user to start a measurement cycle by pressing it once the apparatus 10 is properly clamped onto a leaf. Upon activation, the main controller 32 detects actuation (e.g., button switch) and begins executing the predefined sequence for controlled heating, data acquisition, and analysis.

While the switch 54 provides a convenient option for manual operation, it is not required for system functionality. In alternative embodiments, the test may be initiated automatically through electrical sensing via the electrical sensor 24 or mechanical sensing through a spring tension sensor (e.g., stain gauge or load cell) disposed at the pivot pin 18. In robotic applications, various other detection methods may be used such as vision-based and torque-based sensing that can confirm when a leaf is gripped. This functionality ensures that the apparatus 10 can operate effectively in both handheld and automated robotic implementations, offering flexibility in deployment based on user preference and application requirements.

During operation, the main controller 32 initiates a measurement cycle by activating the heating unit 26 through the heat control unit 36. As the leaf temperature changes, the electrical sensor 24 captures variations in capacitance or resistivity. The signal conditioning unit 34 processes these signals, which are then analyzed by the main controller 32. Simultaneously, the sensor management unit 38 collects environmental data from the environmental sensors 40 to adjust for external influences.

The real-time clock 42 timestamps the data, which is stored in the memory module 44 for later retrieval. If enabled, the GPS module 46 records the location of the measurement. The processed results can be transmitted via the wireless communication unit 48, allowing remote users to access the data in real-time. Power is supplied and regulated by the power management system 52, ensuring efficient operation. Together, these components allow the apparatus 10 to perform real-time, non-invasive monitoring of plant leaf water content, providing valuable insights for precision irrigation and plant health assessment.

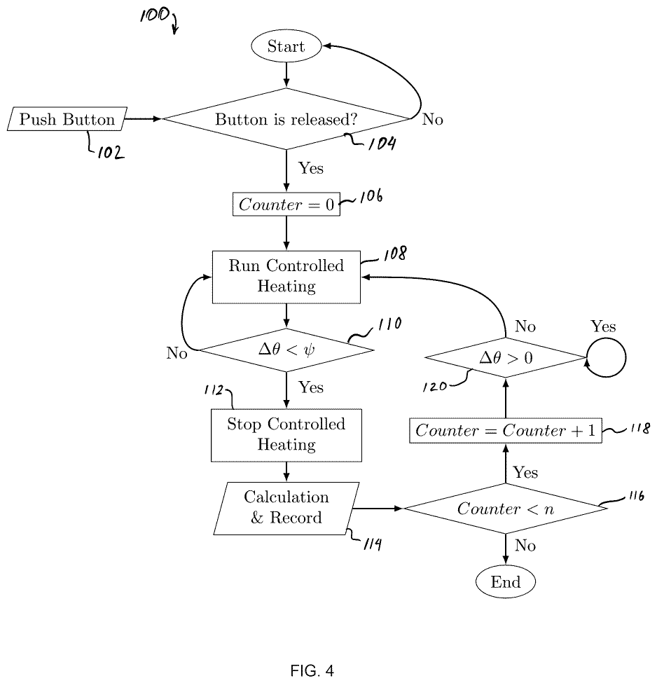

Referring to FIG. 4, a flowchart illustrates a method 100 executed by the main controller 32 for controlling the operation of the apparatus 10 to monitor plant leaf water content. The method may be embodied as software instructions stored in memory module 44 and executable by the main controller 32. The method begins with step 102, where the apparatus 10 awaits user input, for example, via a push button. Once the push button is pressed, the method proceeds to step 104, where the apparatus 10 determines whether the button has been released. If the button has not been released, the apparatus 10 remains in a waiting state. If the button is released, the method continues to step 106, where a counter variable is initialized to zero, ensuring that measurement cycles are accurately tracked.

In other aspects, instead of requiring a push button to initiate the method in step 102, the process may begin automatically when the apparatus 10 detects that a leaf has been clamped between the jaws 14a, 14b. This can be achieved through electrical sensing via the electrical sensor 24, which detects contact with a leaf and signals the main controller 32 to initiate the measurement cycle. Alternatively, a mechanical sensor or a spring tension sensor pin 18 can determine when the clamp 12 is partially or fully closed around an object, triggering the start of the process, as described above. In embodiments, robotic automated systems may be used to detect when the leaf is grasped and to activate the apparatus 10 in response to the confirmation that a leaf is grasped. This automated initiation eliminates the need for manual input, allowing for seamless operation in both handheld and robotic implementations. Once a leaf is detected, the method proceeds directly to step 108, where the apparatus 10 confirms engagement and begins the measurement sequence.

In step 108, the apparatus 10 initiates controlled heating of the leaf sample using the heating unit 26. The main controller 32 regulates the heating process based on predefined parameters. The system then moves to step 110, where it continuously monitors whether the temperature change (Δθ) of the leaf sample is less than a threshold value (ψ). If the temperature change remains below the threshold, the apparatus 10 maintains heating and continues monitoring. Once the temperature change reaches the threshold value, the method proceeds to step 112, where the apparatus 10 stops controlled heating to prevent excessive heat application and potential damage to the leaf.

Following the cessation of heating, the method advances to step 114, where the apparatus 10 performs calculations based on the acquired data and records the temperature response and electrical properties of the leaf. This data is stored in the memory module 44 for further processing.

In some embodiments, the process may additionally or alternatively include acquiring an electrical measurement using a capacitance sensor that is coupled to the leaf, and processing the electrical measurement as an indicator of plant water content or hydration. The capacitance sensor may be implemented as one or more electrodes arranged to capacitively couple to the leaf tissue and generate a capacitance-related signal that varies with a dielectric property of the leaf, a moisture level of the leaf, and/or a change in such properties over time. The capacitance sensor may be read by the controller 50 via the sensing unit 20, and the electrical measurement may be time-stamped and stored in the memory module 44 in association with other measurements obtained during the process.

In some embodiments, the capacitance sensor may be read independently from operation of the heating unit 18, for example to obtain a baseline measurement, a periodic passive measurement, and/or a corroborating measurement that is combined with a thermal-response metric derived from temperature measurements. In some embodiments, the capacitance sensor may be configured for wired and/or wireless interrogation, for example by an interrogator unit that energizes the capacitance sensor and receives a response indicative of the capacitance-related signal, and the received response may be used by the controller 50 to update a plant water content estimate, validate a prior estimate, and/or generate an output, alert, or irrigation control signal.

In step 116, the apparatus 10 evaluates whether the counter value is less than a predefined number of iterations (n), which determines the number of measurement cycles required for an accurate assessment. If the counter value is still below n, the method continues to step 118. If the counter reaches n, the method terminates, and the final measurement data is stored and made available for further analysis.

At step 118, the counter is incremented by one to track the number of completed measurement cycles. At step 120, the method checks whether the measured temperature change (Δθ) is greater than zero, to determine if a heating cycle has reached its end. If the temperature change is not greater than zero, the method loops back to repeat the heating process to step 108. This method ensures controlled and repeatable data acquisition, enabling precise and reliable assessment of plant leaf water content based on both thermal and electrical measurements. The iterative nature of the process enhances measurement accuracy by capturing multiple data points before concluding the analysis.

The heat control unit 36 may also adjust heating parameters based on readings from the environmental sensors 40. In particular, the temperature change threshold (Δθ) used to determine when heating should cease may be dynamically modified in response to ambient conditions such as air temperature, humidity, and atmospheric pressure. For example, in high ambient temperatures, the leaf may require less additional heat to reach a given Δθ, prompting the heat control unit 36 to reduce the applied heating power or shorten the heating duration to prevent overheating. Conversely, in lower ambient temperatures, the apparatus 10 may compensate by increasing heating duration or power to achieve a reliable thermal response. Similarly, humidity levels affect leaf transpiration rates and thermal conductivity; therefore, in high-humidity environments, the apparatus 10 may modify the Δθ threshold to ensure accurate hydration assessment. Additionally, changes in atmospheric pressure can influence heat dissipation, particularly at higher elevations. The heat control unit 36 may factor in pressure variations and adjust heating accordingly to maintain consistency in data acquisition across different environments. By integrating environmental sensor feedback, the heat control unit 36 enhances measurement precision, ensuring that thermal response readings accurately reflect the leaf's hydration status rather than being skewed by external environmental factors.

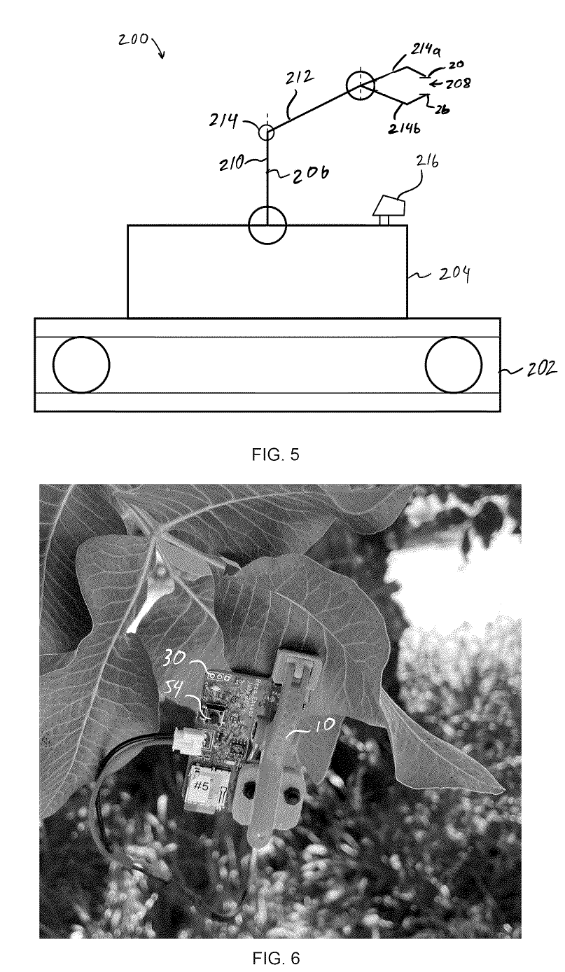

Referring to FIG. 5, a mobile robotic platform 200 is shown, which is configured to transport a moisture sensing grasper for monitoring plant leaf water content. The robotic platform 200 is a self-propelled system designed to navigate agricultural environments autonomously or under remote control. The robotic platform 200 includes a tracked or wheeled system 202, a base 204, and an articulating arm 206 that extends from the base to support and maneuver the sensing apparatus.

The track or wheel system 202 is configured to provide mobility across varied terrain, including fields, orchards, and vineyards. The system 202 may include continuous tracks for improved traction or wheeled drive mechanisms for more efficient maneuverability. The base 204 serves as the structural housing for key components, including the control unit 30, which governs the operation of the robotic platform, articulating arm, and sensing unit.

Extending from the base 204 is the articulating arm 206, which is configured to position a grasper end effector 208 in close proximity to a plant leaf for sensing operations. The articulating arm 206 may include one or more articulated links 210, 212, each interconnected by rotational joints 214 that allow for multi-axis movement. The arm's movement may be controlled by servo motors, hydraulic actuators, or electromechanical actuators, enabling precise positioning of the grasper end effector 208 relative to target plant structures.

The grasper end effector 208 is mounted at the distal end of the articulating arm 206 and includes a pair of opposing jaws 214a, 214b, which are configured to clamp onto a plant leaf for measurement. These jaws 214a, 214b are actuated using motor-driven linkages, cable mechanisms, or pneumatic actuators, providing controlled gripping force to ensure secure contact with the leaf while minimizing damage. The grasper end effector 208 integrates the sensing unit 20 on jaw 214a and the heating unit 26 on jaw 214b, allowing for simultaneous thermal and electrical measurements when clamped onto the leaf.

The articulating arm 206 may incorporate various degrees of freedom, allowing for movements such as extension, retraction, rotation, and elevation adjustments to adapt to different plant heights and orientations. Sensors such as force-torque sensors, encoders, and vision-based guidance systems may be included to enhance the system's ability to autonomously identify and engage leaves with minimal user intervention.

The mobile robotic platform 200 of FIG. 5 may further include a navigation and detection system 216 to enable autonomous movement, plant identification, and precise positioning of the articulating arm 206 for engaging plant leaves. The navigation and detection system 216 may include vision-based navigation, LiDAR, depth cameras, ultrasonic sensors, and GPS-based localization, which collectively enhance the platform's ability to navigate through agricultural environments, detect plant structures, and automatically initiate the measurement process.

The navigation and detection system 216 may utilize RGB cameras, multispectral cameras, or hyperspectral imaging to identify plant leaves based on their shape, size, and spectral reflectance properties. This allows the navigation and detection system 216 to differentiate between plant species and assess leaf positioning for optimal sensor engagement. The LiDAR system, in conjunction with stereoscopic or depth cameras, provides three-dimensional mapping of the surrounding environment, enabling precise distance measurement and object detection. This is particularly useful for avoiding obstacles, aligning the articulating arm 206 with plant targets, and dynamically adjusting movement based on terrain conditions.

Additionally, the robotic platform 200 may include GPS-based navigation to autonomously follow pre-defined paths within an orchard, vineyard, or greenhouse. The robotic platform 200 may further integrate inertial measurement units (IMUs) and wheel encoders for dead reckoning, ensuring accurate position tracking even in GPS-denied environments, such as dense canopy areas.

To facilitate automated plant engagement, the robotic platform 200 may incorporate machine learning algorithms and artificial intelligence (AI)-based object recognition models to identify suitable leaves for measurement. Once a target leaf is detected, the main controller 32 processes the information and sends commands to the articulating arm 206 to position the grasper end effector 208 around the leaf. When the robotic platform 200 determines that the leaf has been securely clamped, either through electrical sensing via the electrical sensor 24 or mechanical sensing at the pivot pin 18, it automatically initiates the measurement process as described in FIG. 4, without requiring manual intervention.

By integrating these advanced navigation and detection technologies, the robotic platform 200 can autonomously traverse fields, locate plant leaves, and execute precise, non-invasive water content measurements, significantly enhancing efficiency and scalability in precision agriculture applications.

The demand for an affordable, efficient, and non-invasive sensor for measuring plant water stress is substantial, particularly in drought-prone regions where water conservation is a critical concern. With regulatory frameworks such as the Sustainable Groundwater Management Act (SGMA) placing increasing pressure on agricultural producers to improve irrigation efficiency and reduce water consumption, technologies that enable real-time water stress monitoring have become increasingly valuable. By providing immediate and accurate data on plant hydration levels, the disclosed sensor facilitates precise irrigation management, helping farmers conserve water, lower operational costs, and comply with regulatory requirements. This capability is particularly beneficial for high-value crops, where effective water management is essential for maintaining productivity and profitability. Given the growing need for sustainable agricultural solutions, the potential for widespread adoption of this technology is considerable.

Beyond regulatory compliance and water conservation, the disclosed sensor offers several key commercial advantages. The strong market demand for precision irrigation tools underscores the necessity of a solution that improves water use efficiency while remaining cost-effective. By reducing water waste and optimizing irrigation schedules, the sensor directly addresses these needs. Its affordability and ease of integration into existing agricultural practices further enhance its scalability, making it suitable for widespread implementation across various farming operations. Additionally, the ability to rapidly and non-invasively assess plant water status provides a distinct competitive advantage over traditional, more invasive methods. Companies investing in this technology can position themselves as leaders in the agricultural technology sector, offering an innovative solution that meets the evolving demands of modern farming.

This disclosure presents a significant advancement in plant water stress monitoring, offering a practical and commercially viable solution for precision irrigation. By addressing the limitations of existing technologies and providing a scalable, cost-effective alternative, the disclosed sensor stands to play a transformative role in improving agricultural water management and sustainability.

From the foregoing, it will be appreciated that specific embodiments of the disclosure have been described herein for purposes of illustration, but that various modifications may be made without deviating from the scope of the disclosure. Accordingly, the disclosure is not limited except as by the appended claims. The following Examples illustrate embodiments of the present disclosure. These Examples are intended to be illustrative only and are not intended to limit the scope of the present disclosure.

### EXAMPLES

### Example 1

This example describes using the describes the use of the apparatus 10 for measuring plant leaf water content in a field environment.

As shown in the photograph of FIG. 6, the apparatus was clamped onto a plant leaf, allowing the apparatus 10 to collect real-time hydration data. The apparatus included a spring-loaded clamp, which applied a consistent gripping force to hold the leaf securely between the jaws. The sensing unit, integrated on the first jaw, made direct contact with the leaf surface to measure thermal and electrical properties. Opposing the sensing unit, the heating unit was mounted on the second jaw, providing controlled heat pulses for temperature-based hydration assessment.

The control unit 30, attached to the apparatus, controlled system operation and data acquisition. The switch 54, located on the control unit, was used for manual initiation of the measurement process. Once activated, the heating unit applied a controlled thermal pulse to the leaf while the sensing unit recorded the heat transfer rate and electrical properties. The collected data was processed by the main controller on the control unit 30, which analyzed hydration levels based on temperature changes and capacitance variations. Environmental factors such as ambient temperature, humidity, and pressure, measured by the environmental sensors, were also considered to improve data accuracy.

The processed hydration data was stored in the memory module and later retrieved for analysis. The wireless communication unit transmitted the data to a remote server or mobile device, allowing for real-time monitoring of plant hydration levels. The system also used GPS to geotag measurements, providing spatial mapping of plant water content across the field.

Alternate embodiments may be devised without departing from the spirit or the scope of the present technology. Additionally, well-known elements of embodiments of the systems, apparatuses, and methods have not been described in detail or have been omitted so as not to obscure the relevant details of the systems, apparatuses, and methods.

The terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises ... a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

When the terms “coupled” and “connected,” along with their derivatives, are used, these terms are not intended as synonyms for each other. For example, “connected” may be used to indicate that two or more elements are in direct physical or electrical contact with each other. “Coupled” may mean that two or more elements are in direct physical or electrical contact (e.g., directly coupled) or that two or more elements are not in direct contact with each other but yet still cooperate or interact with each other (e.g., indirectly coupled).

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, top/bottom, and proximal/distal. Such descriptions are merely used to facilitate the discussion and are not intended to restrict the application of disclosed embodiments. Various operations may be described as multiple discrete operations in tum, in a manner that may be helpful in understanding embodiments; however, the order of description should not be construed to imply that these operations are order dependent.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

Various embodiments of the systems, apparatuses, and methods have been described, and in many of the different embodiments many features are similar. To avoid redundancy, repetitive description of these similar features may not be made in some circumstances. It shall be understood, however, that description of a first-appearing feature applies to the later described similar feature and each respective description, therefore, is to be incorporated therein without such repetition.

What is claimed is:

1. An apparatus for measuring water content of a plant, comprising:
a clamp including a pair of opposing jaws, the jaws configured to receive and secure a leaf of the plant therebetween;
a sensing unit disposed on a first jaw of the pair of opposing jaws, the sensing unit configured to measure at least one thermal and electrical property of the leaf;
a heating unit disposed on a second jaw of the pair of opposing jaws, the heating unit configured to apply controlled heat to the leaf;
a control unit operably coupled to the sensing unit and the heating unit, the control unit configured to:
regulate power to the heating unit to achieve a target heat application profile,
receive data from the sensing unit corresponding to a temperature change and an electrical property of the leaf, and
analyze the received data to determine the water content of the plant.

2. The apparatus of claim 1, wherein the clamp includes a spring-loaded mechanism to maintain a consistent clamping force on the leaf.

3. The apparatus of claim 1, wherein the sensing unit includes a temperature sensor which is at least one of a thermocouple, a resistance temperature detector (RTD), or a thermistor.

4. The apparatus of claim 1, wherein the sensing unit further includes an electrical sensor which is at least one of a capacitance sensor, a resistive sensor, or an impedance sensor.

5. The apparatus of claim 1, wherein the heating unit further includes a resistive heating element.

6. The apparatus of claim 1, wherein the control unit further includes:
an electrical battery;
a power management system configured to regulate electrical power from the battery to the heating unit and the sensing unit;
a memory module configured to store the collected data; and
a real-time clock configured to timestamp measurement data.

7. The apparatus of claim 6, wherein the control unit further includes a wireless communication unit configured to transmit the collected data to a remote computing device.

8. The apparatus of claim 1, further comprising at least one environmental sensor configured to monitor at least one environmental parameter include at least one of ambient temperature, humidity, atmospheric pressure, wind speed, or light intensity.

9. The apparatus of claim 8, wherein the control unit is further configured to adjust heating parameters based on the at least one environmental parameter.

10. The apparatus of claim 1, wherein the control unit is further configured to compare the measured thermal and electrical responses of the leaf to predetermined thresholds to determine the water content of the plant.

11. A method for determining a water content of a plant, comprising:
securing a leaf of the plant between a pair of opposing jaws of a spring-loaded clip;
initiating a controlled heating cycle by supplying power to a heating unit disposed on one jaw of the pair of opposing jaws;
monitoring a temperature change in the leaf using a temperature sensor disposed on another jaw of the pair of opposing jaws;
determining whether the temperature change has reached a predefined threshold;
discontinuing heating upon detecting that the temperature change has reached the predefined threshold;
recording the temperature response of the leaf;
measuring an electrical property of the leaf using an electrical sensor;
storing the recorded temperature response and the measured electrical property and environmental data in a memory module; and
analyzing the stored data to determine the water content of the plant.

12. The method of claim 11, further comprising:
determining whether the temperature change is greater than zero; and
stopping the controlled heating cycle in response to the temperature change being less than zero.

13. The method of claim 11, further comprising:
timestamping the recorded temperature response and electrical property using a real-time clock.

14. The method of claim 11, further comprising:
geotagging the recorded temperature response and electrical property using a GPS module.

15. A mobile robotic platform for measuring a water content of a plant, comprising:
a mobility system configured to navigate an agricultural environment, the mobility system comprising at least one of a tracked system or a wheeled system;
a base housing a control unit configured to process measurement data and to control platform operation;
an articulating arm extending from the base, the articulating arm comprising one or more links interconnected by rotational joints, the articulating arm configured to position a sensing apparatus relative to a leaf of the plant; and
a grasper end effector for measuring properties of the leaf, the apparatus including:
a pair of opposing jaws, the jaws configured to receive and secure the leaf therebetween;
a sensing unit disposed on a first jaw of the pair of opposing jaws, the sensing unit configured to measure at least one thermal and electrical property of the leaf;
a heating unit disposed on a second jaw of the pair of opposing jaws, the heating unit configured to apply controlled heat to the leaf; and
a control unit operably coupled to the sensing unit and the heating unit, the control unit configured to:
regulate power to the heating unit to achieve a target heat application profile,
receive data from the sensing unit corresponding to a temperature change and an electrical property of the leaf, and
analyze the received data to determine the water content of the plant.

16. The mobile robotic platform of claim 15, wherein the sensing unit includes a temperature sensor which is at least one of a thermocouple, a resistance temperature detector (RTD), or a thermistor.

17. The mobile robotic platform of claim 15, wherein the sensing unit further includes an electrical sensor which is at least one of a capacitance sensor, a resistive sensor, or an impedance sensor.

18. The mobile robotic platform of claim 15, wherein the heating unit include a resistive heating element.

19. The mobile robotic platform of claim 15, wherein the control unit further includes:
an electrical battery;
a power management system configured to regulate electrical power from the battery to the heating unit and the sensing unit;
a memory module configured to store the collected data; and
a real-time clock configured to timestamp measurement data.

20. The mobile robotic platform of claim 19, wherein the control unit further comprises a wireless communication unit configured to transmit the collected data to a remote computing device.

21. The mobile robotic platform of claim 15, further comprising at least one environmental sensor configured to monitor at least one environmental parameter include at least one of ambient temperature, humidity, atmospheric pressure, wind speed, or light intensity.

22. The mobile robotic platform of claim 21, wherein the control unit is further configured to adjust heating parameters based on the at least one environmental parameter.

23. The mobile robotic platform of claim 15, wherein the control unit is further configured to compare the measured thermal and electrical properties of the leaf to predetermined thresholds to determine the water content of the plant.
